(19) United States
(12) Patent Application Publication
(10) Pub. No.: US 2023/0270816 A1
(43) Pub. Date: Aug. 31, 2023

(54) COMBINATION OF ANTINEOPLASTIC ANTIBIOTICS AND BCL-2 INHIBITORS FOR THE TREATMENT OF NPM-1-DRIVEN ACUTE MYELOID LEUKEMIA (AML)

(71) Applicants: INSERM (INSTITUTE NATIONAL DE LA SANTÉ ET DE LA RECHERCHE MÉDICALE), Paris (FR); UNIVERSITÉ PARIS CITÉ, Paris (FR); CENTRE NATIONAL DE LA RECHERCHE SCIENTIFIQUE (CNRS), Paris (FR)

(72) Inventors: Hugues BLAUDIN DE THE, Paris (FR); Hsin Chieh WU, Paris (FR)

(21) Appl. No.: 18/004,338

(22) PCT Filed: Jul. 5, 2021
(86) PCT No.: PCT/EP2021/068554

§ 371 Date: Jan. 5, 2023

(30) Foreign Application Priority Data
Jul. 6, 2020 (EP) 20305760.9

(51) Int. Cl. A61K 38/12; A61K 31/635; A61P 35/00

(52) U.S. Cl. CPC A61K 38/12; A61K 31/635; A61P 35/00

(57) ABSTRACT

Acute myelogenous leukaemia (AML) often bear a mutation in the NPM1 nucleolar chaperone, but the transforming properties of the NPM1c oncoprotein remain incompletely understood. Here the inventors show that NPM1c binding to PML, a key senescence gene, disrupts PML nuclear bodies (NB) yielding proliferation, mitochondrial alterations and intracellular stress. Actinomycin-D (ActD), an anticancer antibiotic with clinical efficacy in NPM1c-AMLs, targets these dysfunctional mitochondria to induce ROS. The later disrupt disulphide-linked NPM1c/PML complex, restoring PML NBs and initiating senescence. An ActD-responsive patient displayed features of mitochondria-initiated senescence. These studies highlight unexpected mitochondrial involvement both downstream of the NPM1c/PML axis and as a key feature of ActD therapy. More particularly, the inventors pretreated AML cells with ActD and/or Venetoclax, a Bc12-targeting agent and showed that the two drugs sharply synergized to abolish clonogenic growth of NPM1c-expressing, but not control or PML-deficient cells. Collectively, these results support that combination of antineoplastic antibiotics and BCL-2 inhibitors would be suitable for the treatment of NPM-1-driven acute myeloid leukemia (AML).

### FIELD OF THE INVENTION

The present invention is in the field of medicine, in particular oncology.

### BACKGROUND OF THE INVENTION

Acute myeloid leukemia (AML) is a genetically heterogeneous disease, with a highly variable prognosis and an overall high mortality rate. The 5-year overall survival of adult AML patients is less than 50%, and only 20% of elderly patients survive over 2 years. Cytogenetic alterations classify AML into three risk based-categories: favorable, intermediate and unfavorable. Patients with normal karyotype belong to the intermediate risk category and their prognosis is determined by specific genetic alterations, particularly Nucleophosmin-1 (NPM-1) mutation and FMS-like tyrosine kinase-3 (FLT-3) internal tandem duplication (ITD).

The NPM1 nucleolar chaperone has a broad range of activities, from ribosome biogenesis to control of Myc or TP53 signalling^{1,2}. In 30% of acute myelogenous leukaemia (AML), highly clustered NPM1 mono-allelic mutations yield frameshifts that create de novo nuclear export signals³. While the resulting NPM1c oncoprotein is not the initiating AML mutation, its continuous expression is required for maintenance of established AMLs, where it downregulates TP53 signalling and sustains high expression of Hox genes^{4,5}. PML nuclear bodies (NBs) exert pro-senescent and tumour suppressive functions by enhancing TP53 and Rb activities⁶⁻⁹. PML NBs also control mitochondrial fitness^{10,11}. PML NBs are directly implicated in eradication of acute promyelocytic leukaemia (APL) by retinoic acid or arsenic therapy¹².

### SUMMARY OF THE INVENTION

The present invention is defined by the claims. In particular the present invention relates to combination of antineoplastic antibiotics and BCL-2 inhibitors for the treatment of NPM-1-driven acute myeloid leukemia (AML).

### DETAILED DESCRIPTION OF THE INVENTION

Acute myelogenous leukaemia (AML) often bear a mutation in the NPM1 nucleolar chaperone, but the transforming properties of the NPM1c oncoprotein remain incompletely understood. Here the inventors show that NPM1c binding to PML, a key senescence gene, disrupts PML nuclear bodies (NB) yielding proliferation, mitochondrial alterations and intracellular stress. Actinomycin-D (ActD), an anticancer antibiotic with clinical efficacy in NPM1c-AMLs, targets these dysfunctional mitochondria to induce ROS. The later disrupt disulphide-linked NPM1c/PML complex, restoring PML NBs and initiating senescence. An ActD-responsive patient displayed features of mitochondria-initiated senescence. Other anticancer antibiotics similarly activate this mitochondrial, ROS, PML, TP53 pathway. These studies highlight unexpected mitochondrial involvement both downstream of the NPM1c/PML axis and as a key feature of ActD therapy. More particularly, to explore any interference between ActD and other mitochondria-targeting drugs, the inventors pre-treated AML cells with ActD and/or Venetoclax, a Bc12-targeting agent. Remarkably, these two drugs sharply synergized to abolish clonogenic growth of NPM1c-expressing, but not control or PML-deficient cells. Collectively, these results support that combination of antineoplastic antibiotics and BCL-2 inhibitors would be suitable for the treatment of NPM-1-driven acute myeloid leukemia (AML).

The first object of the present invention relates to a method of treating NPM-1-driven acute myeloid leukemia (AML) in a subject in need thereof comprising administering to the subject a therapeutically effective combination comprising an antineoplastic antibiotic and a BCL-2 inhibitor.

As used herein, the term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells.

As used herein the term “NPM-1” has its general meaning in the art and refers to nucleophosmin-1 (which may also be referred to as also known as N038, nucleolar phosphoprotein B23, numatrin, or NPM-1).

As used herein the term “NPM-1 mutation” refers to any mutation that could occur in NPM-1 and that is associated with AML progression. The mutations are present in the coding regions.

Any NPM-1 mutation is encompassed by the invention, including point mutations, inversion, translocations, deletions, frame shifts . . . Exemplary mutations are described in the literature (e.g. B. Falini, C. Mecucci, E. Tiacci, M. Alcalay, R. Rosati, L. Pasqualucci et al. Cytoplasmic nucleophosmin in acute myelogenous leukemia with a normal karyotypeN Engl J Med, 352 (2005), pp. 254-266) and are encompassed in the invention. Mutations in NPM-1 may be identified by any suitable method in the art, but in some embodiments the mutations are identified by one or more of polymerase chain reaction, sequencing, histochemical stain for NPM-1 localization, as well as immunostaining method using anti-mutant-NPM-1 antibody.

According to the present invention, the term “NPM-1-driven acute myeloid leukemia” has the same meaning than the term “acute myeloid leukemia (AML) with mutated NPM-1”.

In some embodiments, the present invention relates to a method of treating a resistant NPM-1-driven acute myeloid leukemia (AML) in a subject in need thereof comprising administering to the subject a therapeutically effective combination comprising an antineoplastic antibiotic and a BCL-2 inhibitor.

As used herein, the term “resistant NPM-1-driven acute myeloid leukemia (AML)” denotes a NPM-1-driven AML that is totally or partially insensitive to therapeutic drugs, thus leading to a drug resistance. Drug resistance may be a primary drug resistance or an acquired drug resistance.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]). In particular, in the case of AMLs, maintenance therapy may eradicate clinically invisible minimal residual disease.

In particular, the method of the present invention is particularly suitable for inducing growth arrest of AML cells with mutant NPM-1, reducing bone marrow blasts in NPM-1 mutant AML patients and/or correcting the defects in nucleolar organization and function imposed by NPM-1 mutation. In addition, the reformation of PML bodies and activation of TP53, independently triggered by both agents is theoretically predicted to synergize for senescence induction (see EXAMPLE).

As used herein, the term “antineoplastic antibiotic”, also called “anticancer antibiotic” or “antitumour antibiotic”, has its general meaning in the art and refers to any anticancer drug that affects DNA synthesis and replication by inserting into DNA or by donating electrons that result in the production of highly reactive oxygen compounds (superoxide) that cause breakage of DNA strands. Typically said drugs are produced by microorganisms, such as Streptomyces genus.

In some embodiments, the antineoplastic antibiotic is selected from the group consisting of actinomycin D, doxorubicin, daunorubicin, neocarzinostatin, bleomycin, peplomycin, mitomycin C, aclarubicin, pirarubicin, epirubicin, zinostatin stimalamer, and idarubicin.

In some embodiments, the antineoplastic antibiotic is actinomycin D that has the formula of:

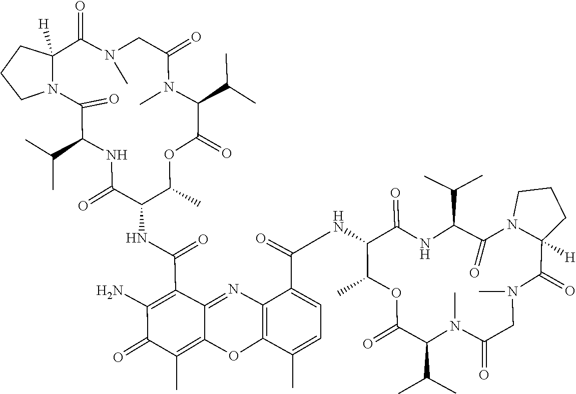 [SMILES: Cc1c2oc3c(C)ccc(C(=O)N[C@@H]4C(=O)N[C@@H](C(C)C)C(=O)N5CCC[C@H]5C(=O)N(C)CC(=O)N(C)[C@@H](C(C)C)C(=O)O[C@@H]4C)c3nc-2c(C(=O)N[C@@H]2C(=O)N[C@@H](C(C)C)C(=O)N3CCC[C@H]3C(=O)N(C)CC(=O)N(C)[C@@H](C(C)C)C(=O)O[C@@H]2C)c(N)c1=O]

As used herein, the term “BCL-2 inhibitor” refers to an agent that is capable of inhibiting one or more proteins in the BCL-2 family of anti-apoptotic proteins, e.g., BCL-2, BCL-xL, and BCL-w. In some embodiments, a BCL-2 inhibitor of the disclosure inhibits one protein of the BCL-2 family selectively, e.g., a BCL-2 inhibitor may selectively inhibit BCL-2 and not BCL-xl or BCL-w. The BCL-2 inhibitor described herein may inhibit one or more of BCL-2, BCL-xL, and BCL-w.

In some embodiments, the inhibitor of BCL-2 anti-apoptotic family of proteins inhibits BCL-2. In some embodiments, the inhibitor of BCL-2 anti-apoptotic family of proteins inhibits BCL-2 and does not inhibit other members of the BCL-2 family of proteins, e.g., does not inhibit BCL-xL or BCL-w. In some embodiments, the BCL-2 inhibitor is a BH3-mimetic.

In some embodiments, a BCL-2 inhibitor interferes with the interaction between the BCL-2 anti-apoptotic protein family member and one or more ligands or receptors to which the BCL-2 anti-apoptotic protein family member would bind in the absence of the inhibitor. In some embodiments, an inhibitor of one or more BCL-2 anti-apoptotic protein family members, wherein the inhibitor inhibits at least one BCL-2 protein specifically, binds only to one or more of BCL-xL, BCL-2, BCL-w and not to other Bcl-2 anti-apoptotic Bcl-2 family members, such as Mcl-1 and BCL2A1.

Binding affinity of a BCL-2 inhibitor for BCL-2 family proteins may be measured. By way of example, binding affinity of a BCL-xL inhibitor may be determined using a competition fluorescence polarization assay in which a fluorescent BAK BI 13 domain peptide is incubated with BCL-xL protein (or other BCL-2 family protein) in the presence or absence of increasing concentrations of the BCL-XL inhibitor as previously described (see, e.g., U.S. Patent Publication 20140005190; Park et al. Cancer Res. 73:5485-96 (2013); Wang et al., Proc. Natl. Acad Sci USA 97:7124-9 (2000); Zhang et al., Anal. Biochem. 307:70-5 (2002); Bruncko et al., J. Med. Chem. 50:641-62 (2007)). Percent inhibition may be determined by the equation: 1−[(mP value of well−negative control)/range)]×100%. Inhibitor}’ constant 0¾) value is determined by the formula: Kj=[I]5o/([h]5o/Ki+[P]o/Ki÷V) as described in Bruncko et al., J. Med. Chem. 50:641-62 (2007) (see, also, Wang, FEBS Lett. 360: 111-114 (1995)).

Examples of BCL-2 inhibitors include ABT-263 (4-[4-[[2-(4-chlorophenyl)-5,5-dimethylcyclohexen-1-yl]methyl]piperazin-1-yl]-N-[4-[[(2R)-4-mo [symbol] holin-4-yl-1-phenylsulfanylbutan-2-yl]amino]-3-(trifluoromethylsulfonyl)phenyl]sulfonylbenzamide or IUPAC, (R)-4-(4-((4′-chloro-4,4-dimethyl-3,4,5,6-tetrahydro-[1,r-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide) {see, e.g., Park et al., 2008, J. Med. Chem. 51:6902; Tse et al., Cancer Res., 2008, 68:3421; International Patent Appl. Pub. No. WO2009/155386; U.S. Pat. Nos. 7,390,799, 7,709,467, 7,906,505, 8,624,027) and ABT-737 ([4-[(4′-Chloro[1,r-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]benzamide, Benzamide, 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]- or 4-[4-[[2-(4-chlorophenyl)phenyl]methyl]piperazin-1-yl]N-[4-[[(2R)-4-(dimethylamino)-1-phenylsulfanylbutan-2-yl]amino]-3-nitrophenyl]sulfonylbenzamide) {see, e.g., Oltersdorf et al., Nature, 2005, 435:677; U.S. Pat. No. 7,973,161; U.S. Pat. No. 7,642,260).

In some embodiments, the BCL-2 inhibitor is a quinazoline sulfonamide compound {see, e.g., Sleebs et al., 2011, J. Med. Chem. 54: 1914). In some embodiments, the BCL-inhibitor is a small molecule compound as described in Zhou et al., J Med. Chem., 2012, 55:4664 {see, e.g., Compound 21 (R)-4-(4 -chlorophenyl)-3-(3-(4-(4-(4-((4-(dimethylamino)-1- (phenylthio)butan-2-yl)amino)-3-nitrophenylsulfonamido)phenyl)piperazin-1-yl)phenyl)-5-ethyl-1-methyl-1H-pyrrole-2-carboxylic acid) and Zhou et al., J Med. Chem., 2012, 55:6149 {see, e.g., Compound 14 (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-ethyl-2-methyl-1H-pyrrole-3-carboxylic acid; Compound 15 (R)-5-(4-Chlorophenyl)-4-(3-(4-(4-(4((4-(dimethylamino)-1-(phenylthio)butan-2-yl)amino)-3-nitrophenylsulfonamido)phenyl)piperazin-1-yl)phenyl)-1-isopropyl-2-methyl-1H-pyrrole-3-carboxylic acid).

In some embodiments, the BCL-inhibitor is a BCL-2/BCL-xL inhibitor such as BM-1074 {see, e.g., Aguilar et al., 2013, J. Med. Chem. 56:3048); BM-957 {see, e.g., Chen et al., 2012, J. Med. Chem. 55:8502); BM-1197 {see, e.g., Bai et al., PLoS One 2014 Jun 5;9(6):e99404. Doi: 10.1371/journal.pone. 009904);ven U.S. Patent Appl. No. 2014/0199234; N-acylsufonamide compounds (see, e.g., Int. Patent Appl. Pub. No. WO 2002/024636, Int. Patent Appl. Pub. No. WO 2005/049593, Int. Patent Appl. Pub. No. WO 2005/049594, U.S. Pat. No. 7,767,684, U.S. Pat. No. 7,906,505). In some embodiments, the BCL-2 inhibitor is a small molecule macrocyclic compound (see, e.g., Int. Patent Appl. Pub. No. WO 2006/127364, U.S. Pat. No. 7,777,076). In some embodiments, the BCL-2 inhibitor is an isoxazolidine compound (see, e.g., Int. Patent Appl. Pub. No. WO 2008/060569, U.S. Pat. No. 7,851,637, U.S. Pat. No. 7,842,815). In some embodiments, the BCL-2 inhibitor is S44563 (see, e.g., Loriot et. al., Cell Death and Disease, 2014, 5, el423). In some embodiments, the BCL-2 inhibitor is (R)-3-((4′-chloro-[1,r-biphenyl]-2-yl)methyl)-N-((4-(((R)-4-(dimethylamino)-1-(phenylthio)butan-2-yl)amino)-3-nitrophenyl)sulfonyl)-2,3,4,4a,5,6-hexahydro-1H-pyrazino[1,2-a]quinoline-8-carboxamide. In another embodiment, the BCL-2 inhibitor is a small molecule heterocyclic compounds (see, e.g.,XJ.S. Pat. No. 9018381).

In some embodiments, the BCL-2 inhibitor is selected from the group consisting of navitoclax, venetoclax, A-1155463, A-1331852, ABT-737, obatoclax, S44563, TW-37, A-1210477, AT101, HA14-1, BAM7, sabutoclax, UMI-77, gambogic acid, maritoclax, MIM1, methylprednisolone, iMAC2, Bax inhibitor peptide V5, Bax inhibitor peptide P5, Bax channel blocker, and ARRY 520 trifluoroacetate.

In some embodiments, the BCL2 inhibitor is venetoclax that has the formula of:

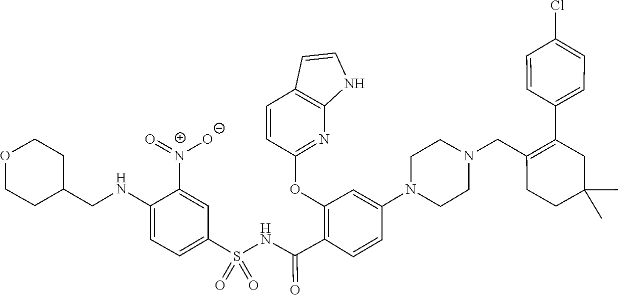 [SMILES: CC1(C)CCC(CN2CCN(c3ccc(C(=O)NS(=O)(=O)c4ccc(NCC5CCOCC5)c([N+](=O)[O-])c4)c(Oc4ccc5cc[nH]c5n4)c3)CC2)=C(c2ccc(Cl)cc2)C1]

As used herein, the term “combination” is intended to refer to all forms of administration that provide a first drug together with a further (second, third . . . ) drug. The drugs may be administered simultaneous, separate or sequential and in any order. Drugs administered in combination have biological activity in the subject to which the drugs are delivered. Typically, the antineoplastic antibiotic (e.g. actinomycin D) is intravenously administered and the BCL-2 inhibitor (e.g. venetoclax) is administered by intravenous route or oral route. According to the invention, the active ingredients of the invention may be administered as a combined preparation for simultaneous, separate or sequential use in the treatment of AML.

By a “therapeutically effective amount” is meant a sufficient amount of the actives ingredients of the invention to treat AML at a reasonable benefit/risk ratio applicable to any medical treatment. It will be understood that the total daily usage of the active ingredients of the invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific active ingredients employed; the duration of the treatment; drugs used in combination or coincidental with the specific active ingredients employed; and like factors well known in the medical arts. For example, it is well within the skill of the art to start doses of the active ingredients at levels lower than those required to achieve the desired therapeutic effect and to gradually increase the dosage until the desired effect is achieved.

The active ingredients of the invention may be combined with pharmaceutically acceptable excipients, and optionally sustained-release matrices, such as biodegradable polymers, to form pharmaceutical compositions. The term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the carrier is a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

The present invention also relates to a kit-of-parts comprising an antineoplastic antibiotic and a BCL-2 inhibitor for use in the treatment of NPM-1-driven acute myeloid leukemia (AML) in a subject in need thereof.

The invention will be further illustrated by the following figures and examples. However, these examples and figures should not be interpreted in any way as limiting the scope of the present invention.

### FIGURES

FIG. 1: Actinomycin D and venetoclax synergize to abolish clonogenic growth of NPM1c-expressing, but not control or PML-deficient cells. Methylcellulose colony formation assays of AML2 cell lines upon 2 hours pre-seeding exposure to Venetoclax and/or ActD. Results are expressed as the mean value of triplicate samples ±SD. Unpaired t test. ***p<0.001.n=3.

FIG. 2: ActD enhances Venetoclax anti-leukaemic effects. Response to ActD and/or Venetoclax in a transplantable AML model initiated by NPM1c+IDH1^{R132H} mutation. A, GFP abundance in bone marrow samples collected at treatment interruption and B, survival curve, n=2. C, abundance of human AML cells in xenografted mice treated for two weeks. Combined therapy induces AML regression.

### EXAMPLE

### NPM1c Alters PML Nuclear Body Formation and Promotes Cell Growth

Leukemic cells harbouring NPM1c mutations exhibit some defects in NB formation, resembling APL microspeckles (data not shown)^{14,15}. Transient NPM1c transfection in cell lines disrupted PML NB. We explored non-leukemic primary HSC from a Flp-inducible humanized Npm1c (variant A) knock-in mouse model (Npm1^{frt-Ca/+,} R26^{FlpoER})³² (data not shown). Four weeks after tamoxifen exposure, these HSCs exhibited dramatic reduction of NBs numbers (data not shown). Similarly, hematopoietic progenitors differentiated from a mouse embryonic stem cell (mESC) NPM1c knock-in model, displayed significantly fewer PML NBs. Transient expression of NPM1c disrupted PML NBs in mouse embryonic fibroblasts (MEFs) stably expressing GFP-PML-III (data not shown). Cysteine 288 in the de novo C-terminal NPM1c sequences is highly redox-sensitive^{16, 17} and has been implicated in nucleolar export and oxidative stress control¹⁶. Remarkably, NPM1c^{C288A} only modestly interacted with PML and no longer delocalized PML NBs (data not shown). Conversely, an exquisitely redox-sensitive PML C389 residue¹⁸ was required for efficient NPM1c/PML interaction and NB-disruption (data not shown). Immunoprecipitation experiments demonstrated that ectopically expressed NPM1c tightly interacts with PML, a process that requires NPM1c C288 and PML C389, but not a PML cysteine residue implicated in arsenic binding⁴⁴ (data not shown). We determined whether a disulfide bond might govern interaction of NPM1c with PML. For this, we transiently transfected PML and NPM1 variants and purified His-PML proteins under denaturing conditions, followed by NPM1 Western blot analyses, in the absence or presence of the protein reducing-agent TCEP. In NPM1c-transfected cells, high molecular weight species reactive to PML and NPM1c antibodies were observed, only when NPM1c C288 and PML C389 were both present and protein reduction reagent omitted (data not shown), suggesting that a disulphide-mediated NPM1c/PML interaction impairs NB assembly and initiates micro-speckle formation. However, while NPM1c binding is required for PML NB disruption, cytoplasmic localization of NPM1c did not require PML, as observed in NMP1c knock-in Pml^{-/ -}mESCs (data not shown).

To explore possible functional consequences of NPM1c/PML interactions, we then stably expressed GFP-NPM1-derived fusions in leukemia-derived AML2 cells where NPM1 gene is wild-type. In this isogenic system, expression of NPM1c blunted NB formation while Npm1 c^{C288S} did not (data not shown). Similar results were found in an embryonic stem cell (ESC) knock-in model in the undifferentiated state (data not shown) or after enforcing their differentiation towards hematopoietic progenitors (data not shown). NPM1c cytoplasmic localisation does not require PML, as demonstrated by PML silencing in AML3 cells that constitutively express NPM1c- or knock-in of NPM1c in Pml^{-/-}ESC (data not shown). Stable expression of NPM1c, but not NPM1c^{C288S}, decreased the basal level TP53 (data not shown), increased clonogenic activity in methyl-cellulose cultures (data not shown) and sharply activated transcription of E2F or Myc target genes (data not shown). NPM1c expression also downregulated expression of wild-type NPM1 and ARF levels, suggesting that the oncogenic mutation may aggravate NPM1 haplo-insufficiency¹⁹ (data not shown). Low PML expression was confirmed in primary AML patient sample (data not shown) and could independently amplify NPM1c-driven defects of PML-NBs formation, contributing to AML pathogenesis.

### NPM1 Impacts Mitochondria and Drives Stress Response

Since PML NBs may control mitochondrial fitness^{10,11}, and NPM1c impairs NB-biogenesis, we assessed the impact of NMP1c expression on mitochondrial status. In tamoxifen-treated NPM1c knock-in mice³², we found an increase in mitochondria number in phenotypic HSC and LSK (Lin⁻Scal⁺Kit⁺) progenitors, but not in Lin-negative cells (data not shown). Similarly, in NPM1c-expressing isogenic AML2 cells, number of mitochondria increased, while branching pattern decreased (data not shown) and cristae were lost, as determined by transmission electron microscopy (data not shown). Accordingly, transcriptomic or proteomic analyses of the isogenic AML2 or ESC knock-in models revealed dysregulation of several mitochondria-related pathways (data not shown). Functionally, these were associated with enhanced production of ROS, mitochondrial superoxides and higher membrane potential, while the overall mitochondrial mass was unchanged (data not shown). Mitochondrial impairments were further substantiated by leakage of mitochondrial DNA to the cytoplasm in AML2-NPM1c or knock-in ES cells (mESCs) (data not shown). NPM1c expression was associated with decreased expression of electron transport chain proteins, such as complex II, reflecting transcriptional downregulation of SDH genes (data not shown).

Mechanistically, PGC1A (peroxisome proliferator-activated receptor gamma coactivator 1-alpha) and TFAM (transcription factor A, mitochondrial), two key regulators mitochondrial protein genes, showed decreased expression in AML2-NPM1c or OCI-AML3 cells (hereafter referred as AML3, which are NPM1c mutant; data not shown). TFAM reduction may reflect transcriptional down-regulation of PGC1A by IFN signalling¹⁶. Importantly, stable NPM1c expression drove PGC1α hyperacetylation resulting in its functional inactivation, possibly through PML NB downregulation (data not shown)¹⁰. These altered mitochondria and release of mtDNA into the cytoplasm activate multiple downstream intracellular stress pathway, particularly IFN and NFKB targets (data not shown), at least in part through cGAS (cyclic GMP-AMP synthase) activation and cGAMP production (data not shown). Other stress pathways, such as oxidative stress or unfolded protein response were also activated (data not shown), resulting in decreased protein synthesis (data not shown). Western blot analyses comparing AML2-NPM1c to AML2-NPM1 cells validated activation of multiple stress pathways by the NPM1c oncogene, including the up-regulation of key TP53-repressed metabolic enzymes (data not shown). As expected, these mitochondrial alterations yielded decreased ATP/ADP+AMP or GTP/GDP+GMP ratios, reflecting metabolic stress (data not shown). Stress responses could directly or indirectly favour leukaemogenesis²⁰⁻²². Functionally, activation of these stress pathways could favour AML cell fitness⁴⁵ Indeed, inhibition of cGAS activity by G140 selectively suppressed growth of AML2-NPM1c cells (data not shown), while inhibition of JAK1/2 signalling by CYT387 inhibited growth, more potently in AML2-NPM1c than in AML2-NPM1 cells (data not shown). These mitochondrial defects may constitute targetable intrinsic weaknesses and, for example, account for the exquisite clinical sensitivity of NPM1c-AMLs to Bcl2 antagonists²³. Collectively, our results indicate that NPM1c expression elicits mitochondrial stress, an important determinant for therapeutic response.

### Actinomycin D (ActD) Targets Mitochondria and Promotes Superoxide Response

Half of NPM1c-AML patients have exhibited dramatic responses to single agent ActD (B. F. & M. P. M, unpublished)^{13,24}. Ex vivo, low doses of ActD (5 nM, comparable to patients' concentrations), triggered rapid TP53 activation in AML3, but not AML2 cells (data not shown). Nonetheless, we only observed a small increase in L11/HDM2 interaction in AML3 cells ex vivo or in patient in vivo, suggesting that ribosomal checkpoint activation upon inhibition of rRNA synthesis may not solely account for TP53 activation (data not shown)²⁵. Unexpectedly, ActD treatment fragmented the mitochondrial network in AML3 and NPM1c-ES cells, as early as 1h (data not shown). Biochemically, ActD further inhibited the basal low level of complex II (but not I or IV) activity, primarily in NPM1c-expressing cells (data not shown). ActD did not affect transcription of mtDNA (data not shown), but massively decreased abundance of ribosomal proteins, independently of NPM1c (data not shown). Then, inhibition of electron chain function was followed by ROS production, loss of mitochondrial membrane potential and induction of superoxide transcriptional response and down-regulation of mtDNA through its leakage to the cytoplasm, all preferentially in AML3 and/or AML2-NPM1c-cells (data not shown). Mitochondrial dysfunction upon ActD exposure exacerbated pre-existing NPM1c-driven metabolic stress response, as revealed by decreased ATP level, increased AMP-K or eIF2α phosphorylation (data not shown). Accordingly, a remarkable similarity was found between genes induced by NPM1c and those activated by ActD in AML2 cells (data not shown). Thus, ActD targets mitochondrial function, thereby amplifying its basal disorder in NPM1c-AMLs.

Consequently, ActD-triggered induction of stress pathways may be functionally more significant in NPM1c-expressing cells where their basal activation levels are already high. Similarly, ActD exacerbated pre-existing NMP1c-driven metabolic stress, as revealed by decreased ATP levels and increased AMPK (AMP-activated protein kinase) phosphorylation (data not shown). Collectively, these observations suggest that ActD targets mitochondria, particularly those primed by NPM1c-expression.

### Actinomycin D Activates a ROS/PML/TP53 Senescence Axis in NPM1c-Expressing Cells Ex Vivo

We then explored the cellular impact of mitochondrial poisoning by ActD. Remarkably, ActD treatment disrupted NPM1c-PML adducts, yielding very rapid NB reformation in multiple NPM1c expression models, such as AML2-NPM1c, AML3 cells or NPM1c-transfected MEFs (data not shown). Importantly, PML NB restoration was also observed in primary NPM1c-AML blasts treated ex vivo with ActD (data not shown). In transiently transfected cells, NB restoration by ActD was associated (and likely caused) by disruption of NPM1-PML complexes by high ROS (data not shown). Similarly, in AML3 cells, ActD (as TCEP) restored the normal size of high molecular weight NPM1 or PML species (data not shown). Apart from disrupting NPM1c/PML adducts, ActD-induced ROS may also directly enforce PML biogenesis^{8,44}. Accordingly, NB-restoration was completely blocked by the ROS scavengers N-acetyl cysteine and glutathione in AML2-NPM1c cells (data not shown).

PML NBs are tightly linked to senescence induction. In NPM1c-positive AML3 cells, ActD-driven PML NB reformation was associated to multiple signs of senescence, including TP53 activation, loss of clonogenic activity, increase in p21 or Serpine-1 expression, cGAMP production, and SA-β-Gal activity, suggestive for ActD-driven senescence (data not shown). In keeping with the key role of PML in senescence induction, these features were abolished or attenuated by PML or TP53 deletion (data not shown). Indeed, ActD activates typical transcriptional stress signatures (UPR, ROS signalling), activation of innate immunity and apoptosis/senescence (Myc and E2F down, TP53, IFN, UPR, TNFA or TGFB up, data not shown). While responses were often shared between the two isogenic cell-lines, TGFB and TP53 activation were particularly pronounced in AML2-NPM1c cells. Critically, activation of senescence markers by ActD was blocked by PML excision and/or pre-incubation with NAC/GSH antioxidant (data not shown). Mitochondrial depletion abolished the ability of ActD to induce TP53 and p21 activation, as well as clonogenic activity (data not shown). Collectively, these experiments establish a central role of a ROS/PML/TP53 axis in driving ActD-triggered senescence of AML3 cells.

To demonstrate the essential role of mitochondria in immediate TP53 and p21 activation by ActD, we depleted mitochondria from AML3 cells by growing them in rotenone or antimycin²⁶. Mitochondrial depletion abolished early ActD-triggered TP53 activation (data not shown). Conversely, we used Thenoyl-trifluoroacetone (TTFA) a well-characterized inhibitor of mitochondrial complex II and major inducer of ROS. In AML3 cells, TTFA massively induced mitochondrial superoxide and decreased clonogenic activity (data not shown). In NPM1c-transfected stable GFP-PML-III MEFs, TTFA restored NB formation (data not shown). Collectively, these experiments establish the key role of NPM1c-primed mitochondrial ROS in ActD-driven activation of the PML senescence checkpoint ex vivo.

### ActD Exerts AML-Specific Growth Suppression In Vivo

We first assessed ActD response in primary cells. With our ActD low doses and scheduling, no significant TP53 stabilization was observed in normal bone marrow progenitors in vivo or ex vivo, as well as in human or mouse primary fibroblasts (WI-38, MEFs) ex vivo, (data not shown). However, in two NPM1c-AML immune-deficient mice xenografted with primary human NPM1c-AML blasts, ActD rapidly triggered selective stabilization of human TP53, but not its Trp53 mouse counterpart (data not shown), highlighting tumor-specific targeting. ActD therapy dramatically reduced the leukemic burden after 5 days (data not shown), while the few remaining human AML cells now expressed differentiated features (c-Kit loss, CD11b or CD14 induction, data not shown). Similarly, in a murine NPM1c-driven AML model²⁷, leukaemia-selective in vivo TP53 activation was accompanied by AML regression and blast differentiation (data not shown). Finally, in AML3 xenografts, PML was required for ActD triggered TP53 stabilization and anti-leukemic effect (data not shown), all strengthening conclusions from our ex vivo studies. Collectively, these in vivo observations suggest that ActD favors PML-dependant TP53 activation and growth arrest in NMP1c-AML blasts but (at least initially) spares normal cells.

We then explored AML cells from a patient who exhibited a dramatic 10⁴-fold blast decrease after a single ActD course of daily ActD injections for 5 days^{13,24} (data not shown). In leukemic blasts sampled from the peripheral blood, in vivo reformation of PML NBs started at 6 h, was complete by 12 h and was accompanied by TP53 stabilisation and target gene activation, including P21 (data not shown). While cGAS very rapidly aggregated after ActD (data not shown), no significant increase in L11/HMD2 interaction was detected up to 48 h, while γH2AX expression/foci only appeared at 48h (data not shown), arguing against potent early activation of ribosomal or DNA-damage checkpoints in vivo. Thus, PML NB-reformation is an immediate response of AML cells to ActD therapy in vivo. Remarkably, pathway analyses of transcriptomes from AML-rich peripheral blood revealed immediate and massive acute stress responses (expression of HSP1A, FOS, EGR1) and activation of PML NBs as early as 6 hours following initiation of therapy (data not shown). We also observed immediate shutoff of the NFκB pathway, including extinction of IL8 expression, a distinct feature of mitochondria dysfunction-driven senescence (data not shown)²⁶. Yet, despite repeated daily ActD administrations, these transcriptional changes rapidly normalized (data not shown), pointing to the existence of major adaptive control mechanisms (data not shown). Nevertheless, ActD treatment and mitochondrial stress were followed by some features of senescence (PML NBs, TP53 and P21 up, E2F down) up to 48 h (data not shown). TUNEL-positive apoptotic cells were also detected 48 hours post-treatment and accompanied by a progressive, but massive, inactivation of proliferation genes (data not shown), indicating the co-occurrence of apoptosis. A consolidation course of ActD treatment given when the patient had no residual leukemic blasts was not accompanied by significant TP53 target activation or modulation of stress responses in normal blood cells (data not shown). Collectively, our findings support the idea that ActD-driven acute mitochondrial stress initiates PML-NB-activated senescence/apoptosis in NPM1c AML cells in vivo.

### ActD Potentiates Venetoclax Anti-Leukemic Effects

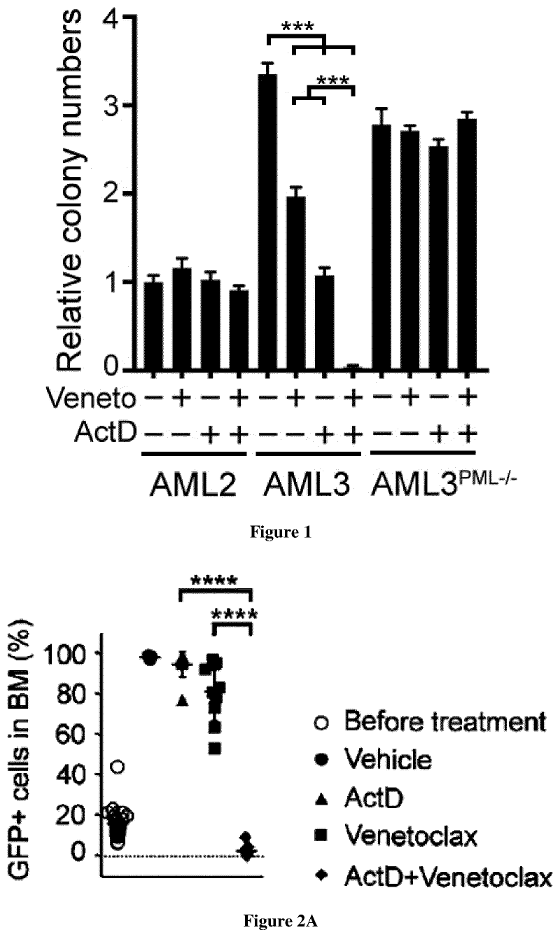

To explore any interference between ActD and other mitochondria-targeting drugs, we pre-treated AML cells with ActD and/or Venetoclax, a Bc12-targeting agent (FIG. 1).

Having demonstrated NPM1c-driven complex II impairment (data not shown) and ActD-mediated mitochondrial toxicity (data not shown), we hypothesized that ActD might enhance the anti-AML activity of Venetoclax. As expected, in AML3 cells, Venetoclax induced mitochondrial fragmentation, reduction of mitochondrial membrane potential, production of ROS and leakage of mtDNA to the cytoplasm. Critically, all of these features were strongly potentiated by co-treatment with ActD (data not shown). Accordingly, in AML2-NPM1c, Venetoclax and ActD strongly synergized to activate cGAS activity and PML NB reformation (data not shown). Remarkably, these two drugs sharply synergized to abolish clonogenic growth of NPM1c-expressing, but not control or PML-deficient cells (FIG. 1). Collectively, ex vivo, the ActD/Venetoclax combination synergizes for mitochondrial targeting in NPM1c-expressing cells, yielding PML-dependant growth arrest.

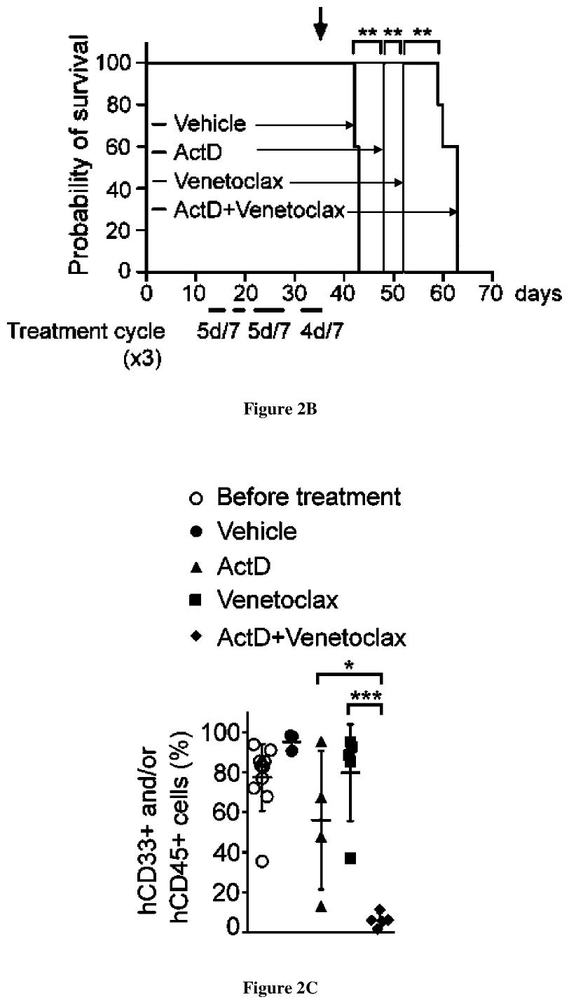

We therefore explored the ActD/Venetoclax interaction in different NPM1c-driven AML models in vivo. First, in murine NPM1c-driven AMLs²⁷ the combination treatment showed lower leukemic burden and enhanced blast differentiation (data not shown). Second, in a double conditional knock-in of NPM1c plus IDH1^{R132H}, only the combined treatment eliminated AML cells from the bone marrow and yielded a significant survival advantage (FIG. 2A, B). Finally, in the xenograft model described above, ActD and Venetoclax (but neither agent alone) cleared AML cells from the bone marrow (FIG. 2C). Thus, ActD and Venetoclax dramatically synergized to clear AMLs in vivo.

### Discussion:

We demonstrate that NPM1c binds the PML tumour suppressor, at least in part through disulphide bounds, driving defective PML NB formation. Several NPM1c-driven pro-leukemic phenotypes unravelled here (TP53 silencing, mitochondrial dysfunction, enhanced ROS or INF signalling) were reported in Pml^{-/-}cells and may reflect defective NB formation^{8,16}. Our studies highlight the critical role of a highly redox sensitive NPM1c-specific cysteine (C288) in PML-binding and nucleolar export, reminiscent of C275 implicated in nucleolar targeting of normal Npm1^{16,17,29}. Multiple PML splice variants that cooperate for senescence induction³⁰ precludes direct functional exploration of PML C389 role. NPM1c-driven mitochondrial defects and resulting integrated stress response³¹ could play a critical role in the shift from DNMT3A or IDH1/2 immortalized stem cells to full blown leukemia, through cell autonomous mechanisms³²⁻³⁴ and/or remodelling of the microenvironment²².

Targeting mitochondrial function by antibiotics was proposed as a therapeutic option for cancer^{35,36} and mitochondrial status modulates chemotherapy response^{37,38}. Actinomycin D drives mitochondrial alterations prior to detectable activation of the ribosomal or DNA damage checkpoints. Actinomycin D inhibits complex II activity, which may reflect its structural similarity with FAD, one of its electron transporters. This may explain how ActD induces ROS, immediate early response genes, sensitizes cells to apoptosis or initiates immunogenic cell death³⁹. Complex II is important in hematopoietic progenitors⁴⁰, and regulated by PML⁴¹, contributing to ActD therapeutic index in AML blasts. In vivo, ActD triggered features of mitochondria-induced senescence²⁶. Downstream of acute mitochondrial stress, our experiments reveal a key role of ROS signalling and PML/TP53 senescence (data not shown), drawing an unexpected similarity with acute promyelocytic leukemia, where NBs reformation drive response to arsenic therapy^{12,42}. In that respect, complex II poisoning has activity in APL models⁴³. Doxorubicin triggered immediate mitochondrial rounding, PML-dependent, NPM1c-enhanced and NAC-reversible TP53 activation (data not shown), suggesting that the pathway outlined here may be broadly shared with other anticancer antibiotics. Clinically, as both antineoplastic antibiotics (e.g. ActD) and BCL-2 inhibitors (e.g. Venetoclax) have remarkable clinical activity in NPM1c-AMLs, the dramatic synergy outlined here should rapidly be evaluated in patients.

### REFERENCES

Throughout this application, various references describe the state of the art to which this invention pertains. The disclosures of these references are hereby incorporated by reference into the present disclosure.
1 Heath, E. M. et al. Biological and clinical consequences of NPM1 mutations in AML. Leukemia 31, 798-807, doi:10.1038/leu.2017.30 (2017).
2 Kunchala, P., Kuravi, S., Jensen, R., McGuirk, J. & Balusu, R. When the good go bad: Mutant NPM1 in acute myeloid leukemia. Blood Rev 32, 167-183, doi:10.1016/j.blre.2017.11.001 (2018).
3 Falini, B. et al. Altered nucleophosmin transport in acute myeloid leukaemia with mutated NPM1: molecular basis and clinical implications. Leukemia 23, 1731-1743, doi:10.1038/leu .2009.124 (2009).
4 Cheng, K. et al. The leukemia-associated cytoplasmic nucleophosmin mutant is an oncogene with paradoxical functions: Arf inactivation and induction of cellular senescence. Oncogene 26, 7391-7400, doi:10.1038/sj.onc.1210549 (2007).
5 Brunetti, L. et al. Mutant NPM1 Maintains the Leukemic State through HOX Expression. Cancer Cell 34, 499-512 e499, doi:10.1016/j.ccell.2018.08.005 (2018).
6 Lallemand-Breitenbach, V. & de The, H. PML nuclear bodies: from architecture to function. Curr Opin Cell Biol 52, 154-161, doi:10.1016/j.ceb.2018.03.011 (2018).
7 Hsu, K. S. & Kao, H. Y. PML: Regulation and multifaceted function beyond tumor suppression. Cell Biosci 8, 5, doi:10.1186/s13578-018-0204-8 (2018).
8 Niwa-Kawakita, M. et al. PML is a ROS sensor activating p53 upon oxidative stress. J Exp Med 214, 3197-3206, doi:10.1084/jem.20160301 (2017).
9 Ivanschitz, L. et al. PML IV/ARF interaction enhances p53 SUMO-1 conjugation, activation, and senescence. Proc Natl Acad Sci U S A 112, 14278-14283, doi:10.1073/pnas.1507540112 (2015).
10 Carracedo, A. et al. A metabolic prosurvival role for PML in breast cancer. The Journal of clinical investigation 122, 3088-3100, doi:10.1172/JCI62129 (2012).
11 Ito, K. et al. A PML-PPAR-delta pathway for fatty acid oxidation regulates hematopoietic stem cell maintenance. Nat Med 18, 1350-1358, doi:10.1038/nm.2882 (2012).
12 de The, H., Pandolfi, P. P. & Chen, Z. Acute Promyelocytic Leukemia: A Paradigm for Oncoprotein-Targeted Cure. Cancer Cell 32, 552-560, doi:10.1016/j.ccell.2017.10.002 (2017).
13 Falini, B., Brunetti, L. & Martelli, M. P. Dactinomycin in NPM1-Mutated Acute Myeloid Leukemia. N Engl J Med 373, 1180-1182, doi:10.1056/NEJMc1509584 (2015).
14 El Hajj, H. et al. Retinoic acid and arsenic trioxide trigger degradation of mutated NPM1, resulting in apoptosis of AML cells. Blood 125, 3447-3454, doi:10.1182/blood-2014-11-612416 (2015).
15 Martelli, M. P. et al. Arsenic trioxide and all-trans retinoic acid target NPM1 mutant oncoprotein levels and induce apoptosis in NPM1-mutated AML cells. Blood 125, 3455-3465, doi:10.1182/blood-2014-11-611459 (2015).
16 Huang, M. et al. Role of cysteine 288 in nucleophosmin cytoplasmic mutations: sensitization to toxicity induced by arsenic trioxide and bortezomib. Leukemia 27, 1970-1980, doi:10.1038/leu.2013 .222 (2013).
17 Mukherjee, H. et al. Interactions of the natural product (+)-avrainvillamide with nucleophosmin and exportin-1 Mediate the cellular localization of nucleophosmin and its AML-associated mutants. ACS Chem Biol 10, 855-863, doi:10.1021/cb500872g (2015).
18 Weerapana, E. et al. Quantitative reactivity profiling predicts functional cysteines in proteomes. Nature 468, 790-795, doi:10.1038/nature09472 (2010).
19 Sportoletti, P. et al. Npm1 is a haploinsufficient suppressor of myeloid and lymphoid malignancies in the mouse. Blood 111, 3859-3862, doi:10.1182/blood-2007-06-098251 (2008).
20 Cai, X. et al. Runx1 Deficiency Decreases Ribosome Biogenesis and Confers Stress Resistance to Hematopoietic Stem and Progenitor Cells. Cell Stem Cell 17, 165-177, doi:10.1016/j.stem.2015.06.002 (2015).
21 Milanovic, M. et al. Senescence-associated reprogramming promotes cancer stemness. Nature 553, 96-100, doi:10.1038/nature25167 (2018).
22 Mendez-Ferrer, S. et al. Bone marrow niches in haematological malignancies. Nat Rev Cancer, doi:10.1038/s41568-020-0245-2 (2020).
23 DiNardo, C. D. et al. Molecular patterns of response and treatment failure after frontline venetoclax combinations in older patients with AML. Blood 135, 791-803, doi:10.1182/blood.2019003988 (2020).
24 Beziat, G. et al. Dactinomycin in acute myeloid leukemia with NPM1 mutations. Eur J Haematol, doi:10.1111/ejh.13438 (2020).
25 Bursac, S. et al. Mutual protection of ribosomal proteins L5 and L11 from degradation is essential for p53 activation upon ribosomal biogenesis stress. Proc Natl Acad Sci U S A 109, 20467-20472, doi:10.1073/pnas.1218535109 (2012).
26 Wiley, C. D. et al. Mitochondrial Dysfunction Induces Senescence with a Distinct Secretory Phenotype. Cell Metab 23, 303-314, doi:10.1016/j.cmet.2015.11.011 (2016).
27 Mallardo, M. et al. NPMc+ and FLT3_ITD mutations cooperate in inducing acute leukaemia in a novel mouse model. Leukemia 27, 2248-2251, doi:10.1038/leu.2013.114 (2013).
28 Rossi, S. L. et al. Identification of early gene expression changes in primary cultured neurons treated with topoisomerase I poisons. Biochem Biophys Res Commun 479, 319-324, doi:10.1016/j.bbrc.2016.09.068 (2016).
29 Yang, K. et al. A redox mechanism underlying nucleolar stress sensing by nucleophosmin. Nat Commun 7, 13599, doi:10.1038/ncomms13599 (2016).
30 Bischof, O. et al. Deconstructing PML-induced premature senescence. Embo J 21, 3358-3369 (2002).
31 Costa-Mattioli, M. & Walter, P. The integrated stress response: From mechanism to disease. Science 368, doi:10.1126/science.aat5314 (2020).
32 Loberg, M. A. et al. Sequentially inducible mouse models reveal that Npm1 mutation causes malignant transformation of Dnmt3a-mutant clonal hematopoiesis. Leukemia 33, 1635-1649, doi:10.1038/s41375-018-0368-6 (2019).
33 Gachet, S. et al. Deletion 6q drives T-cell leukemia progression by ribosome modulation. Cancer Discov, doi:10.1158/2159-8290.CD-17-0831 (2018).
34 Yamashita, M., Dellorusso, P. V., Olson, O. C. & Passegue, E. Dysregulated haematopoietic stem cell behaviour in myeloid leukaemogenesis. Nat Rev Cancer, doi:10.1038/s41568-020-0260-3 (2020).
35 Martinez-Outschoorn, U. E., Peiris-Pages, M., Pestell, R. G., Sotgia, F. & Lisanti, M. P. Cancer metabolism: a therapeutic perspective. Nat Rev Clin Oncol 14, 11-31, doi:10.1038/nrclinonc.2016.60 (2017).
36 Rava, M. et al. Therapeutic synergy between tigecycline and venetoclax in a preclinical model of MYC/BCL2 double-hit B cell lymphoma. Sci Transl Med 10, doi:10.1126/scitranslmed.aan8723 (2018).
37 Gentric, G. et al. PML-Regulated Mitochondrial Metabolism Enhances Chemosensitivity in Human Ovarian Cancers. Cell Metab, doi:10.1016/j.cmet.2018.09.002 (2018).
38 Potter, D. S. & Letai, A. To Prime, or Not to Prime: That Is the Question. Cold Spring Harb Symp Quant Biol 81, 131-140, doi:10.1101/sqb.2016.81.030841 (2016).
39 Humeau, J. et al. Inhibition of transcription by dactinomycin reveals a new characteristic of immunogenic cell stress. EMBO Mol Med, e11622, doi:10.15252/emmm.201911622 (2020).
40 Morganti, C., Bonora, M., Ito, K. & Ito, K. Electron transport chain complex II sustains high mitochondrial membrane potential in hematopoietic stem and progenitor cells. Stem Cell Res 40, 101573, doi:10.1016/j.scr.2019.101573 (2019).
41 Guo, S., Cheng, X., Lim, J. H., Liu, Y. & Kao, H. Y. Control of antioxidative response by the tumor suppressor protein PML through regulating Nrf2 activity. Mol Biol Cell 25, 2485-2498, doi:10.1091/mbc.E13-11-0692 (2014).
42 Ablain, J. et al. Activation of a promyelocytic leukemia-tumor protein 53 axis underlies acute promyelocytic leukemia cure. Nature medicine 20, 167-174., doi:doi: 10.1038/nm.3441 (2014).
43 Dos Santos, G. A. et al. (+)alpha-Tocopheryl succinate inhibits the mitochondrial respiratory chain complex I and is as effective as arsenic trioxide or ATRA against acute promyelocytic leukemia in vivo. Leukemia: official journal of the Leukemia Society of America, Leukemia Research Fund, U.K, doi:10.1038/leu.2011.216 (2011).
44 Jeanne M, Lallemand-Breitenbach V, Ferhi O, Koken M, Le Bras M, Duffort S, et al. PML/RARA oxidation and arsenic binding initiate the antileukemia response of As2O3. Cancer Cell 2010;18(1):88-98 doi S1535-6108(10)00241-2[pii] 10.1016/j.ccr.2010.06.003.
45 van Galen P, Mbong N, Kreso A, Schoof E M, Wagenblast E, Ng S W K, et al. Integrated Stress Response Activity Marks Stem Cells in Normal Hematopoiesis and Leukemia. Cell Rep 2018;25(5):1109-17 e5 doi 10.1016/j.celrep.2018.10.021.

1. A method of treating NPM-1-driven acute myeloid leukemia (AML) in a subject in need thereof comprising administering to the subject a therapeutically effective combination comprising an antineoplastic antibiotic and a BCL-2 inhibitor.

2. The method of claim 1 wherein the antineoplastic antibiotic is selected from the group consisting of actinomycin D, doxorubicin, daunorubicin, neocarzinostatin, bleomycin, peplomycin, mitomycin C, aclarubicin, pirarubicin, epirubicin, zinostatin stimalamer, and idarubicin.

3. The method of claim 1 wherein the antineoplastic antibiotic is actinomycin D.

4. The method of claim 1 wherein the BCL-2 inhibitor is selected from the group consisting of navitoclax, venetoclax, A-1155463, A-1331852, ABT-737, obatoclax, S44563, TW-37, A-1210477, AT101, HA14-1, BAM7, sabutoclax, UMI-77, gambogic acid, maritoclax, MEM, methylprednisolone, iMAC2, Bax inhibitor peptide V5, Bax inhibitor peptide P5, Bax channel blocker, and ARRY 520 trifluoroacetate.

5. The method of claim 1 wherein the BCL2 inhibitor is venetoclax.

6. The method of claim 1 wherein the antineoplastic antibiotic is actinomycin D and the BCL-2 inhibitor is venetoclax.

7. A method of treating a resistant NPM-1-driven acute myeloid leukemia (AML) in a subject in need thereof comprising administering to the subject a therapeutically effective combination comprising an antineoplastic antibiotic and a BCL-2 inhibitor.

8. The method of claim 7 wherein the antineoplastic antibiotic is actinomycin D and the BCL-2 inhibitor is venetoclax.

9. A kit-of-parts comprising an antineoplastic antibiotic and a BCL-2 inhibitor for use in the treatment of NPM-1-driven acute myeloid leukemia (AML) in a subject in need thereof.

10. The kit-of-parts of claim 9 wherein the antineoplastic antibiotic is selected from the group consisting of actinomycin D, doxorubicin, daunorubicin, neocarzinostatin, bleomycin, peplomycin, mitomycin C, aclarubicin, pirarubicin, epirubicin, zinostatin stimalamer, and idarubicin.

11. The kit-of-parts of claim 9 wherein the antineoplastic antibiotic is actinomycin D.

12. The kit-of-parts of claim 9 wherein the BCL-2 inhibitor is selected from the group consisting of navitoclax, venetoclax, A-1155463, A-1331852, ABT-737, obatoclax, S44563, TW-37, A-1210477, AT101, HA14-1, BAM7, sabutoclax, UMI-77, gambogic acid, maritoclax, MIM1, methylprednisolone, iMAC2, Bax inhibitor peptide V5, Bax inhibitor peptide P5, Bax channel blocker, and ARRY 520 trifluoroacetate.

13. The kit-of-parts of claim 9 wherein the BCL2 inhibitor is venetoclax.
